(19) United States
(12) Patent Application Publication
(10) Pub. No.: US 2016/0327554 A1
(43) Pub. Date: Nov. 10, 2016

(54) SENSING METHOD AND SENSING CHIP

(71) Applicant: SIWARD Crystal Technology Co., Ltd., Taichung (TW)

(72) Inventors: Ying-Cheng Hung, Taichung (TW); Chuen-Yuan Hsu, Taichung (TW); Yi-Heui Hsieh, Taichung (TW); Chiao-Wen Huang, Taichung (TW)

(21) Appl. No.: 15/148,124

(22) Filed: May 6, 2016

(30) Foreign Application Priority Data
May 7, 2015 (TW) 104114627

(51) Int. Cl. G01N 33/543; G01N 33/553

(52) U.S. Cl. CPC G01N 33/54386; G01N 33/54346; G01N 33/553

(57) ABSTRACT

A sensing chip for sensing a first organic molecule in a sample is disclosed. The sensing chip includes a substrate, wherein there are a plurality of separated nanometers on the substrate and a plurality of local substrate surfaces among the plurality of separated nanometers; a silane fixed on the plurality of local substrate surfaces; and a second organic molecule fixed on surfaces of the separated nanometers, wherein there is specificity between the second organic molecule and the first organic molecule.

### CROSS REFERENCE TO RELATED APPLICATIONS

The application claims the benefit of Taiwan Patent Application No. 104114627, filed on May 7, 2015, at the Taiwan Intellectual Property Office, the disclosures of which are incorporated herein in their entirety by reference.

### FIELD OF THE INVENTION

The present invention is related to a sensing method and a sensing chip, and more particularly to a sensing method and a sensing chip for sensing an organic molecule in a sample.

### BACKGROUND OF THE INVENTION

There are a variety of sensing methods and sensing devices on the market. Common sensing methods include enzyme-linked immunosorbent assay (ELISA) and common sensing devices include experimental plates.

ELISA is a common sensing method, and it has a multi-year history. There are at least two types of ELISA, wherein in one type of ELISA, the target object is an antigen and in another type, the target object is an antibody. They are discussed as follows.

When the target object is an antigen, ELISA includes the following steps: coating a specific antibody on a plastic plate, wherein the time for coating is about 12-18 hours, and washing away excess antibodies after the completion of the coating; adding the target object to carry out a reaction with the coated antibody, wherein the reaction time is about 0.5-2 hours, and if the target object contains an antigen reactive with the coated antibody, the antigen carrying out a specific binding with the coated antibody on the plastic plate; washing away excess target objects, and then adding an antibody with an enzyme reactive with the antigen to bind with the antigen, wherein the time for binding is about 0.5-2 hours; and washing away excess un-bound antibodies with an enzyme, adding a substrate for the enzyme to carry out a color reaction, wherein the time for the color reaction is about 0.5 hour, and then reading the result of the color reaction (i.e. absorbance (OD value)), wherein it takes about 1-2 days to complete the entire test.

When the target object is an antibody, ELISA includes the following steps: coating a known antigen on a plastic plate, wherein the time for coating is about 12-18 hours, and washing away excess antigens after the completion of the coating; adding the target object to carry out a reaction with the coated antigen, wherein the reaction time is about 0.5-2 hours, and if the target object contains a first antibody reactive with the coated antigen, the first antibody carrying out a specific binding with the coated antigen on the plastic plate; washing away excess target objects, and then adding a second antibody with an enzyme to bind with the first antibody, wherein the time for binding is about 0.5-2 hours; and washing away excess un-bound second antibodies with an enzyme, adding a substrate for the enzyme to carry out a color reaction, wherein the time for the color reaction is about 0.5 hour, and then reading the result of the color reaction (i.e. absorbance (OD value)), wherein it takes about 1-2 days to complete the entire test. Accordingly, ELISA has limitations in sensitivity.

ELISA generally uses experimental plates to carry out the test. However, according to test requirements, chips or other sensing devices can also be used. Conversely, experimental plates and the chips can also be used for sensing methods besides ELISA.

Experimental plates on the market have a variety of structures and materials. For example, they can be divided into 6, 12, 24, 48, 96, 384 and 1536 well plates based on the number of wells, can be divided into a flat bottom, round bottom, V-bottom and Easy-Wash bottom (it has characteristics of both the round well bottom and flat well bottom) plates based on the structure of the bottom, can be divided into polystyrene (PS), polypropylene (PP) and poly(vinyl chloride) (PVC) plates based on the material, can be divided into clear, black, white, black with clear bottom and white with clear bottom plates based on the color, and can be divided into general analysis, cell culture and cell analysis, immunoassay and storage plates based on the use. Most immunoassay plates are polystyrene 96-well plates. The bottom surface of immunoassay plates is usually un-treated or treated to cause benzene rings on the plate surface to produce carboxyl groups and hydroxyl groups to increase the binding capacity with molecules intended to be coated on the plates using irradiation technology.

Sensing chips on the market at present can be divided into microarrays and lab-on-a-chips based on the manufacturing process. Microarrays can be divided into gene chips and protein chips.

In order to improve the sensitivity of the tests, improving existing sensing methods and sensing devices is an important issue to the skilled person in the art.

### SUMMARY

In accordance with one aspect of the present invention, a sensing chip for sensing a first organic molecule in a sample is disclosed. The sensing chip includes a substrate having a plurality of nanoparticles spaced apart thereon and a plurality of local substrate surfaces located among the plurality of nanoparticles; a silane disposed on a specific one the plurality of local substrate surfaces; and a second organic molecule disposed on a surface of a specific one of the plurality of nanoparticles, wherein there is a specificity between the second and the first organic molecules.

In accordance with another aspect of the present invention, a sensing chip for sensing a first organic molecule in a sample is disclosed. The sensing chip includes a substrate having a plurality of nanoparticles spaced apart thereon and a plurality of local substrate surfaces located among the plurality of nanoparticles; a second organic molecule disposed on a surface of a specific one of the plurality of nanoparticles, wherein there is a specificity between the second and the first organic molecules; and a modifier disposed on a specific one of the plurality of local substrate surfaces, and dispelling the second organic molecule away from the specific one of the plurality of local substrate surfaces.

In accordance with a further aspect of the present invention, a sensing method for sensing a first organic molecule in a sample is disclosed. The sensing method includes steps of providing a sensing chip including a substrate, a plurality of spaced apart nanoparticles on the substrate and a plurality of local substrate surfaces located among the plurality of nanoparticles; disposing a silane on a specific one of the plurality of local substrate surfaces, and disposing a second organic molecule on a surface of a specific one of the plurality of spaced apart nanoparticles, wherein the silane enhances an adsorptivity of the second organic molecule to the specific one of the plurality of spaced apart nanoparticles; and there is a specificity between the second and the first organic molecules.

### BRIEF DESCRIPTION OF THE DRAWINGS

FIG. 1 shows the sensing chip according to a first embodiment of the present invention;

FIG. 2 shows the sensing chip according to a second embodiment of the present invention;

FIG. 3(a) shows the sensing method according to a first embodiment of the present invention;

FIG. 3(b) shows the sensing method according to a second embodiment of the present invention;

FIG. 4(a) shows the sensing chips according to a first embodiment and a second embodiment of the present invention;

FIG. 4(b) shows the base elements with a through hole according to a first embodiment and a second embodiment of the present invention;

FIG. 4(c) shows the sensing devices according to a first embodiment and a second embodiment of the present invention;

FIG. 5(a) shows the sensing chips according to a first embodiment and a second embodiment of the present invention;

FIG. 5(b) shows the base elements with a through hole according to a first embodiment and a second embodiment of the present invention;

FIG. 5(c) shows the sensing devices according to a first embodiment and a second embodiment of the present invention;

FIG. 6(a) shows the sensing chips according to a first embodiment and a second embodiment of the present invention;

FIG. 6(b) shows the base elements with a through hole according to a first embodiment and a second embodiment of the present invention;

FIG. 6(c) shows the sensing devices according to a first embodiment and a second embodiment of the present invention;

FIG. 7 shows the frames according to a first embodiment and a second embodiment of the present invention;

FIG. 8 shows the comparison of the sensing device of the present invention with Corning's COR-9018 plate;

FIGS. 9-10 show quantifying the human/mouse transforming growth factor beta 1 by a sandwich ELISA method using eBioscience human/mouse TGF beta 1 ELISA kit (Cat. No. 88-8350-88) with the sensing device of the present invention and Corning's COR-9018 plate;

FIG. 11 shows quantifying the human/mouse transforming growth factor beta 1 using a sandwich ELISA method using a eBioscience human/mouse TGF beta 1 ELISA kit (Cat. No. 88-8350-88) with the sensing device of the present invention and Corning's COR-9018 plate;

FIG. 12 shows quantifying the C-Reactive Protein (CRP) using a sandwich ELISA method using a commercial C-Reactive Protein ELISA kit with the sensing device of the present invention and Corning's COR-9018 plate;

FIG. 13(a) shows carrying out ELISA using the sensing device with a material of a glass substrate having gold nanoparticles, and surfaces of the gold nanoparticles are modified with amino silane;

FIG. 13(b) shows carrying out ELISA using the sensing device with a material of a glass substrate having gold nanoparticles;

FIG. 13(c) shows carrying out ELISA using the sensing device with a material of a glass substrate modified with amino silane directly;

FIG. 13(d) shows carrying out ELISA using the sensing device with a material of a glass substrate only;

FIG. 14(a) shows the concentration detection limitation test of the present invention of carrying out ELISA using the sensing device with a material of a glass substrate having gold nanoparticles, and surfaces of the gold nanoparticles are modified with amino silane at low concentrations;

FIG. 14(b) shows the concentration detection limitation test of the present invention of carrying out ELISA using the sensing device with a material of a glass substrate only at low concentrations.

### DETAILED DESCRIPTION

The present invention will now be described more specifically with reference to the following embodiments. It is to be noted that the following descriptions of preferred embodiments of this invention are presented herein for the purposes of illustration and description only; they are not intended to be exhaustive or to be limited to the precise form disclosed.

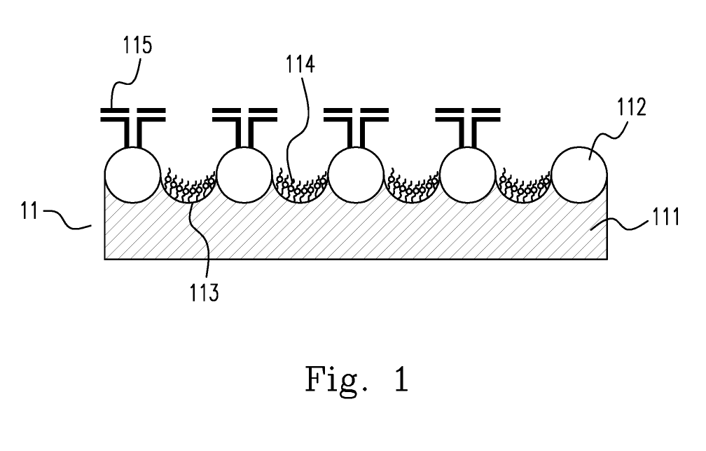

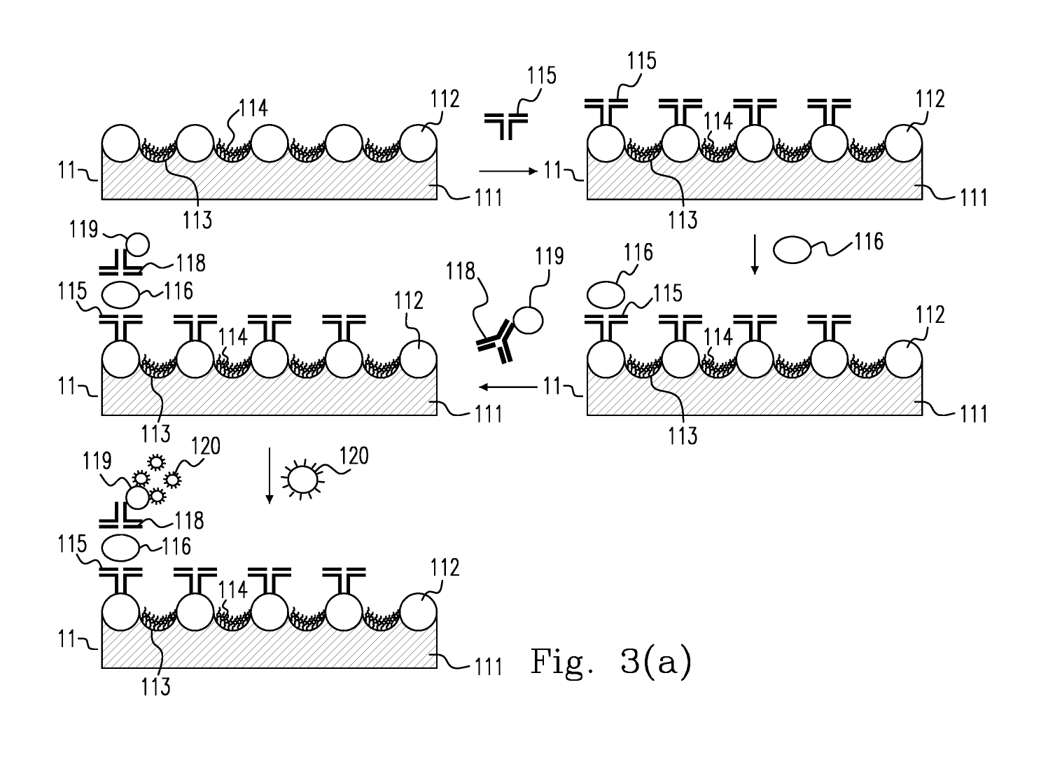

Please refer to FIG. 1 and FIG. 3(a), wherein the first embodiment of the present invention discloses a sensing chip 11 for sensing a first organic molecule 116 in a sample.

The sensing chip 11 includes a substrate 111 having a plurality of nanoparticles 112 spaced apart thereon and a plurality of local substrate surfaces 113 located among the plurality of nanoparticles 112; a silane 114 disposed on a specific one the plurality of local substrate surfaces 113; and a second organic molecule 115 disposed on a surface of a specific one of the plurality of nanoparticles 112, wherein there is a specificity between the second organic molecule 115 and the first organic molecule 116.

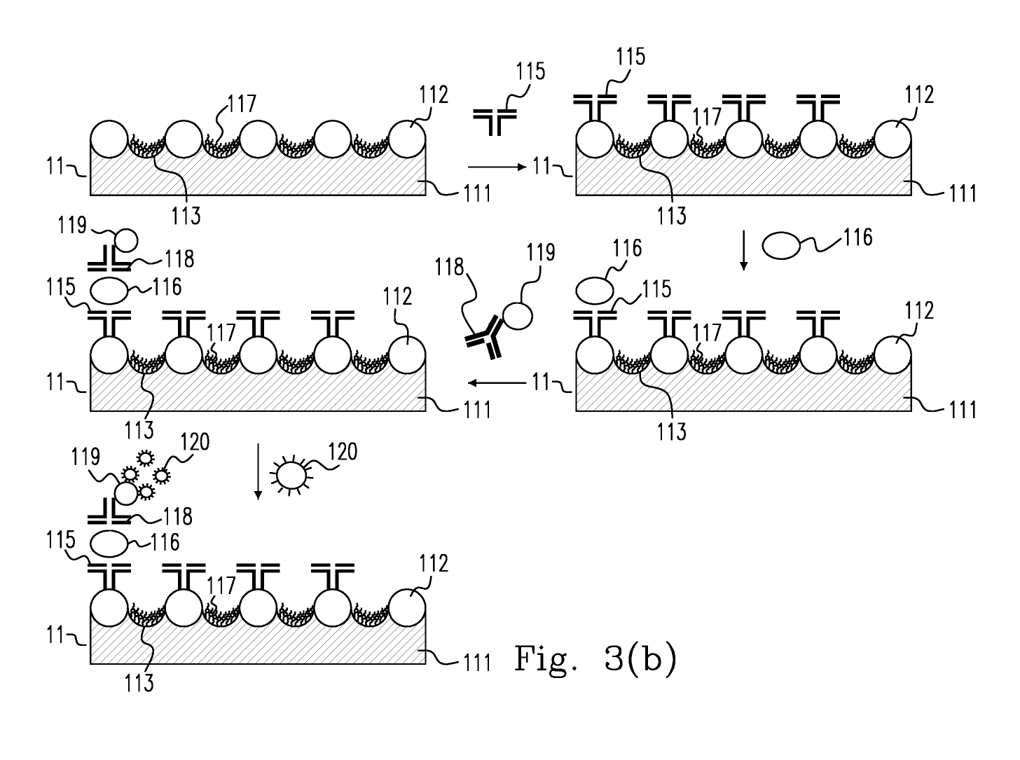

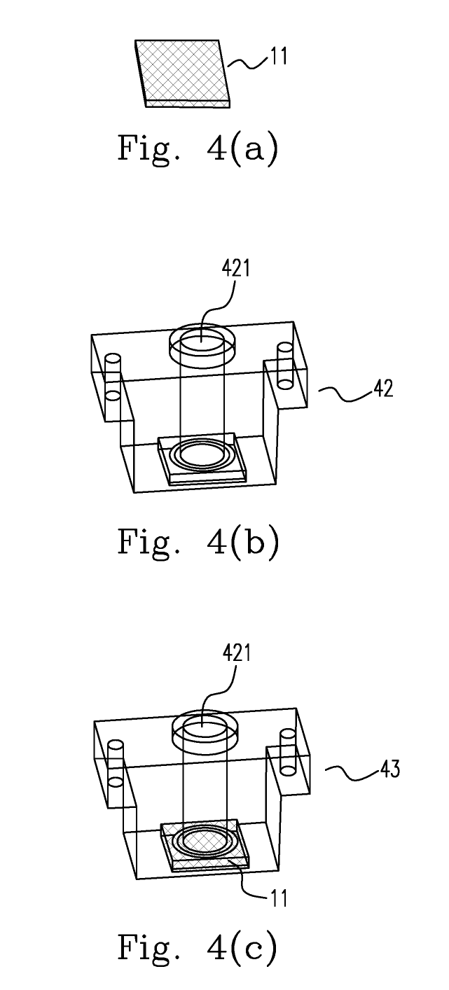

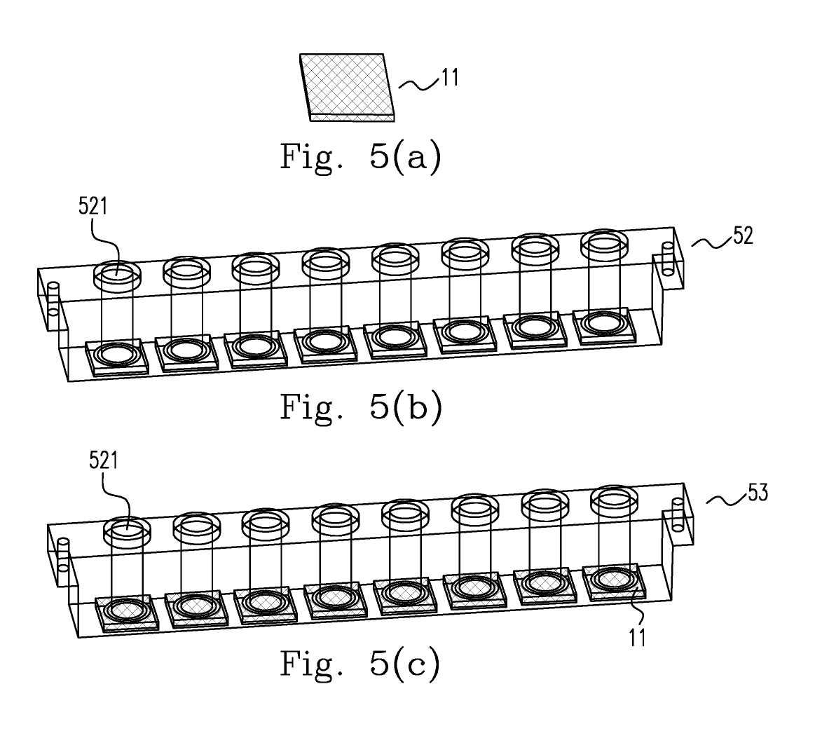

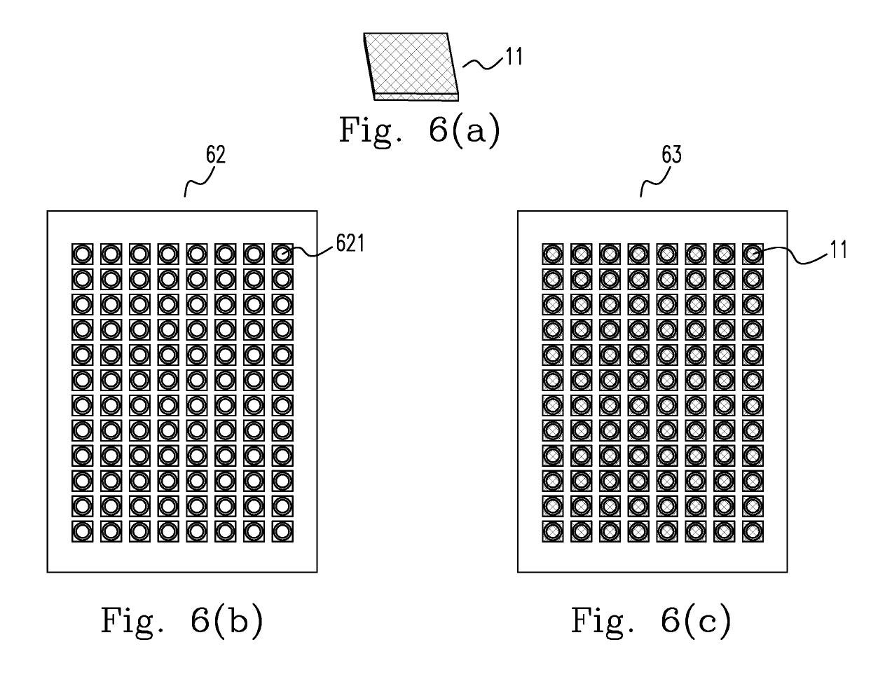

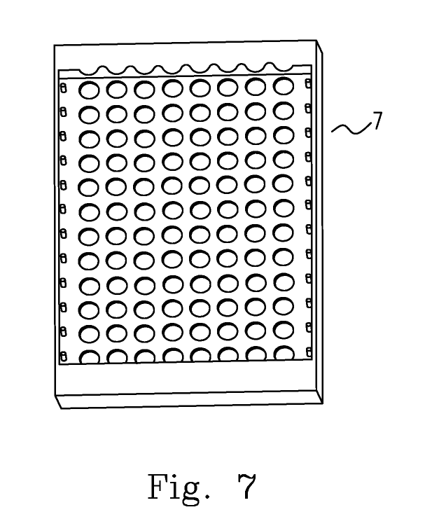

As shown in FIGS. 4-6, the sensing chip 11 in this embodiment may be configured to be detachably disposed on a base element 42, 52, 62 having a through hole 421, 521, 621 for closing the through hole 421, 521, 621 at one end thereof to form a sensing device 43, 53, 63. The sensing device 63 as shown in FIG. 6 may be directly used to sense the first organic molecule 116 in the sample, while the sensing device 43, 53 as shown in FIGS. 4-5 may be further assembled in a frame 7 as shown in FIG. 7 to sense the first organic molecule 116 in the sample. When using the sensing device 43, 53, 63 to sense, any instruments that can be used with experimental plates can be used, such as a spectrometer or automatic microplate washer, wherein the spectrometer may be an ELISA reader.

The sensing chip 11 in this embodiment may be one of a protein chip and a gene chip. The protein chip or the gene chip may be directly used to sense without being combined with other elements.

The second organic molecule 115 may be directly disposed on the surface of the specific one of the plurality of nanoparticles 112. The sample may be but is not limited to blood, urine, cell culture media and other body fluids. The first organic molecule 116 may be a protein, and the protein may be one of an antigen and an antibody. The substrate 111 may be made of a transparent material. The plurality of nanoparticles 112 are made of a metal which is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof. The shape of the nanoparticles is one selected from a group consisting of a circle, island, long strip, triangle, star, annulus, hollow shape, and a combination thereof. The diameter of the nanoparticles may be 1-200 nm. The plurality of local substrate surfaces 113 have a diameter, and the diameter may be 1-100 nm. The silane 114 may be an alkylsilane, aminosilane or other silane. The aminosilane may be a 3-aminopropryltrimethoxysilane (APTMS) or 3-aminopropyltriethoxysilane (APTS). The second organic molecule 115 may be a protein, and the protein may be one of an antigen and an antibody.

The sensing chip 11 or the sensing device 43, 53, 63 in this embodiment may be added with suitable antibodies, standards, buffers, coloring agents, blocking solutions, substrate solutions, and stopping solution, etc. to form a kit.

Please refer to FIG. 1 and FIG. 3(a), wherein the first embodiment of the present invention further discloses a sensing method for sensing a first organic molecule 116 in a sample.

The sensing method includes the following steps. A sensing chip 11 is provided. The sensing chip 11 includes a substrate 111, a plurality of nanoparticles 112 spaced apart on the substrate 111 and a plurality of local substrate surfaces 113 located among the plurality of nanoparticles 112. A silane 114 is disposed on a specific one of the plurality of local substrate surfaces 113. A second organic molecule 115 is disposed on a surface of a specific one of the plurality of nanoparticles 112, wherein there is a specificity between the second organic molecules 115 and the first organic molecules 116.

After the second organic molecule 115 is disposed on the surface of the specific one of the plurality of nanoparticles 112, an Enzyme-linked immunosorbent assay may be performed in the sensing method in this embodiment. That is to say, a sample is added to the through hole 421, 521, 621. After the first organic molecule 116 in the sample specifically binds to the second organic molecules 115, a third organic molecule 118 having an enzyme 119 is added to the through hole 421, 521, 621. After the third organic molecule 118 specifically binds to the first organic molecule 116, a substrate 120 of the enzyme 119 is added to the through hole 421, 521, 621 to carry out a color reaction. A stopping solution is added to stop the color reaction to obtain a result of the color reaction, and a value of the result of the color reaction is read.

As shown in FIGS. 4-6, the sensing method in this embodiment may further configure the sensing chip 11 to be detachably disposed on a base element 42, 52, 62 having a through hole 421, 521, 621 for closing the through hole 421, 521, 621 at one end thereof to form a sensing device 43, 53, 63. The sensing device 63 as shown in FIG. 6 may be directly used to sense the first organic molecule 116 in the sample, while the sensing device 43, 53 as shown in FIGS. 4-5 may be further assembled in a frame 7 as shown in FIG. 7 to sense the first organic molecule 116 in the sample. When using the sensing device 43, 53, 63 to sense, any instruments that can be used with experimental plates can be used, such as a spectrometer or automatic microplate washer, wherein the spectrometer may be an ELISA reader.

The second organic molecule 115 may be directly disposed on the surface of the specific one of the plurality of nanoparticles 112. The sample may be but is not limited to blood, urine, cell culture media and other body fluids. The first organic molecule 116 may be a protein, and the protein may be one of an antigen and an antibody. The substrate 111 may be made of a transparent material. The plurality of nanoparticles 112 are made of a metal which is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof. The shape of the nanoparticles is one selected from a group consisting of a circle, island, long strip, triangle, star, annulus, hollow shape, and a combination thereof. The diameter of the nanoparticles may be 1-200 nm. The plurality of local substrate surfaces 113 have a diameter, and the diameter may be 1-100 nm. The silane 114 may be an alkylsilane, aminosilane or other silane. The aminosilane may be a 3-aminopropryltrimethoxysilane (APTMS) or 3-aminopropyltriethoxysilane (APTS). The second organic molecule 115 may be a protein, and the protein may be one of an antigen and an antibody.

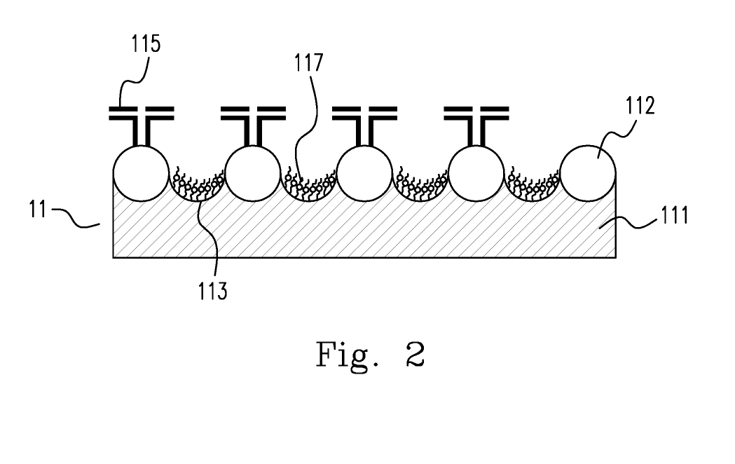

Please refer to FIG. 2 and FIG. 3(b), wherein the second embodiment of the present invention provides a sensing chip 11 for sensing a first organic molecule 116 in a sample.

The sensing chip 11 includes a substrate 111 having a plurality of nanoparticles 112 spaced apart thereon and a plurality of local substrate surfaces 113 located among the plurality of nanoparticles 112; a second organic molecule 115 disposed on a surface of a specific one of the plurality of nanoparticles 112, wherein there is a specificity between the second organic molecules 115 and the first organic molecule 116, and a modifier 117 disposed on a specific one the plurality of local substrate surfaces 113; and a modifier disposed on a specific one of the plurality of local substrate surfaces, and dispelling the second organic molecule 115 away from the specific one of the plurality of local substrate surfaces 113.

As shown in FIGS. 4-6, the sensing chip 11 in this embodiment may be configured to be detachably disposed on a base element 42, 52, 62 having a through hole 421, 521, 621 for closing the through hole 421, 521, 621 at one end thereof to form a sensing device 43, 53, 63. The sensing device 63 as shown in FIG. 6 may be directly used to sense the first organic molecule 116 in the sample, while the sensing device 43, 53 as shown in FIGS. 4-5 may be further assembled in a frame 7 as shown in FIG. 7 to sense the first organic molecule 116 in the sample. When using the sensing device 43, 53, 63 to sense, any instruments that can be used with experimental plates can be used, such as a spectrometer or automatic microplate washer, wherein the spectrometer may be an ELISA reader.

The sensing chip 11 in this embodiment may be one of a protein chip and a gene chip. The protein chip or the gene chip may be directly used to sense without being combined with other elements.

The second organic molecule 115 may be directly disposed on the surface of the specific one of the plurality of nanoparticles 112. The sample may be but is not limited to blood, urine, cell culture media and other body fluids. The first organic molecule 116 may be a protein, and the protein may be one of an antigen and an antibody. The substrate 111 may be made of a transparent material. The plurality of nanoparticles 112 are made of a metal which is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof. The shape of the nanoparticles is one selected from a group consisting of a circle, island, long strip, triangle, star, annulus, hollow shape, and a combination thereof. The diameter of the nanoparticles may be 1-200 nm. The plurality of local substrate surfaces 113 have a diameter, and the diameter may be 1-100 nm. The modifier 117 may be a silane, hydramine or enamine. The silane may be an alkylsilane, aminosilane or other silane. The aminosilane may be a 3-aminopropryltrimethoxysilane (APTMS) or 3-aminopropyltriethoxysilane (APTS). The second organic molecule 115 may be a protein, and the protein may be one of an antigen and an antibody.

The sensing chip 11 or the sensing device 43, 53, 63 in this embodiment may be added with suitable antibodies, standards, buffers, coloring agents, blocking solutions, substrate solutions, and stopping solution, etc. to form a kit.

Please refer to FIG. 2 and FIG. 3(b), wherein the second embodiment of the present invention further provides a sensing method for sensing a first organic molecule 116 in a sample.

The sensing method includes the following steps. A sensing chip 11 is provided. The sensing chip 11 includes a substrate 111, a plurality of nanoparticles 112 spaced apart on the substrate 111 and a plurality of local substrate surfaces 113 located among the plurality of nanoparticles 112. A modifier 117 is disposed on a specific one of the plurality of local substrate surfaces 113, wherein the modifier 117 enhances an adsorptivity of a second organic molecule 115 to the specific one of the plurality of nanoparticles 112. A second organic molecule 115 is disposed on a surface of a specific one of the plurality of nanoparticles 112, wherein there is a specificity between the second organic molecules 115 and the first organic molecule 116.

After the second organic molecule 115 is disposed on the surface of the specific one of the plurality of nanoparticles 112, an Enzyme-linked immunosorbent assay may be performed in the sensing method in this embodiment. That is to say, a sample is added to the through hole 421, 521, 621. After the first organic molecule 116 in the sample specifically binds to the second organic molecules 115, a third organic molecule 118 having an enzyme 119 is added to the through hole 421, 521, 621. After the third organic molecule 118 specifically binds to the first organic molecule 116, a substrate 120 of the enzyme 119 is added to the through hole 421, 521, 621 to carry out a color reaction. A stopping solution is added to stop the color reaction to obtain a result of the color reaction, and a value of the result of the color reaction is read.

As shown in FIGS. 4-6, the sensing method in this embodiment may further configure the sensing chip 11 to be detachably disposed on a base element 42, 52, 62 having a through hole 421, 521, 621 for closing the through hole 421, 521, 621 at one end thereof to form a sensing device 43, 53, 63. The sensing device 63 as shown in FIG. 6 may be directly used to sense the first organic molecule 116 in the sample, while the sensing device 43, 53 as shown in FIGS. 4-5 may be further assembled in a frame 7 as shown in FIG. 7 to sense the first organic molecule 116 in the sample. When using the sensing device 43, 53, 63 to sense, any instruments that can be used with experimental plates can be used, such as a spectrometer or automatic microplate washer, wherein the spectrometer may be an ELISA reader.

The second organic molecule 115 may be directly disposed on the surface of the specific one of the plurality of nanoparticles 112. The sample may be but is not limited to blood, urine, cell culture media and other body fluids. The first organic molecule 116 may be a protein, and the protein may be one of an antigen and an antibody. The substrate 111 may be made of a transparent material. The plurality of nanoparticles 112 are made of a metal which is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof. The shape of the nanoparticles is one selected from a group consisting of a circle, island, long strip, triangle, star, annulus, hollow shape, and a combination thereof. The diameter of the nanoparticles may be 1-200 nm. The plurality of local substrate surfaces 113 have a diameter, and the diameter may be 1-100 nm. The modifier 117 may be a silane, hydramine or enamine. The silane may be an alkylsilane, aminosilane or other silane. The aminosilane may be a 3-aminopropryltrimethoxysilane (APTMS) or 3-aminopropyltriethoxysilane (APTS). The second organic molecule 115 may be a protein, and the protein may be one of an antigen and an antibody.

By using the sensing chip of the present invention for general ELISA, through the nanoparticles disposed on the substrate, the detection signal of the ELISA can be effectively enhanced, and thus the detection limit is reduced to <Ipg/mL.

The method of using the sensing chip of the present invention to perform general ELISA includes steps of first manufacturing a gold nanoparticle array and modifying silane on its surface, coating proteins overnight, and then performing the standard process of detection for general ELISA. It is found that gold nanoparticles can enhance signals and thus improve OD values, so that the distance between dose and response can become wider. The mechanism absorbs proteins through surfaces of the gold nanoparticles and, after being absorbed through the surfaces of the gold nanoparticles, the proteins exhibit three-dimensional structures, and thus the protein structure can be effectively unfolded. From the viewpoint of the adsorption orientation, the proteins have higher specificity. Even at low concentrations, signal points can be effectively differentiated from each other. In addition, modifying silane is a must and used to allow the protein to be coated more evenly, so that at low concentrations, the values among signal points will not change irregularly, and thus the stability of the entire signal is improved. Therefore, the detection limitation can reach 0.064 pg/ml, and the effect of measuring low concentrations can be achieved.

Applying the sensing chip of the present invention for ELISA can coat the protein direct and effective. Surface structure properties of different proteins cause them to adsorb onto the surfaces of the gold nanoparticles through three main forces, and the three main forces are hydrophobic force, electric attraction and ligand bond binding force.

Hydrophobic interaction: In the embodiments of the sensing method and the sensing chip of the present invention, after alkyl silane is absorbed to the substrate between the nanoparticles, the alkyl silane increases the contact angle, the increased contact angle can help protein to become closer to the surfaces of the gold nanoparticles. Once a protein is very close to surface of a gold nanoparticle (the distance between them is less than 1 nm), the hydrophobic region of the protein is likely to contact the hydrophobic region of the gold nanoparticle and bind with it. Therefore, proteins rich in non-polar amino acids (such as tryptophan, valine, leucine, isoleucine or phenylalanine) can achieve strong binding with the surfaces of the gold nanoparticles. Thus, the properties of the end functional group of the alkyl silane really strengthens the protein to adsorb onto the surface of the gold nanoparticle.

Electric attraction: after adsorbing on the substrate, the end NH₂ of an amino silane molecule will form NH₃⁺ in an aqueous solution, wherein the amino silane molecules have a positive charge, and repel the protein with a positive charge. The surface of the gold nanoparticle has a negative charge, and the negative charge attracts a positively charged protein and pulls it close to the surface of the binding region. A protein has a positive charge when the environment pH value is below the isoelectric point of the protein, and therefore a strong attraction occurs with the surface of the gold nanoparticle. Specifically, a lysine and arginine-rich protein region has a large number of positive charges when the environment pH value is below the isoelectric point of lysine (pH 10.4) and the isoelectric point of arginine (pH 12.5). Thus, the positive charge the amino silane molecule carries helps a lysine and arginine-rich protein to adsorb onto the surface of the gold nanoparticle.

Ligand bond binding force: the ligand bond binding force is the strongest of all attraction. A protein contains a large number of sulfur-rich amino acids (cysteine and methionine) which strongly bind to the surface of the gold nanoparticle and, this is because of the attraction between a gold atom (having the ability to conduct electrons) and a sulfur atom (having valence electrons).

Through the three forces, a gold nanoparticle can efficiently adsorb a protein, efficiently enhance the adsorption efficiency of protein, and coat a protein with the minimal protein concentration in the shortest time, and the obtained signal result is better than the general commercial ELISA plate.

Thus, the sensing method and sensing chip of the present invention have advantages of high sensitivity, low cost, and speed. Also, they can detect different antibodies and viruses.

The present invention is applicable to experimental developments such as immunoassay, chemical analysis and enzyme analysis. It is also applicable for the establishment of experimental procedures such as dynamics and temperature control. It is also applicable to antibody identification such as antibody/ligand affinity screening, epitope of monoclonal antibody screening, tumor cell screening and stage identification, anti-idiotypic antibody screening, antibody concentration measurements and fragment screening. It is also applicable to pre-clinical and clinical diagnostics such as bio-marker analysis and point of care. The present invention has wide range of uses.

### Experiments

1. Modifying the sensing chip of the present invention by amino silane to carry out the detection of antigens or antibodies:

First, a hydrophilic modification is made to the substrate surface of the sensing chip of the present invention using oxygen plasma with a weaker energy, and then the substrate is soaked in the (3-Aminopropyl)trimethoxysilane (APTMS) solution such that (3-Aminopropyl)trimethoxysilane binds to the portions of the substrate without nanoparticles. Then, adding antibodies (antigens) directly can cause antibodies (antigens) to be coated on the nanoparticles. Finally, sample (antigens (antibodies)) are added to carry out the detection test for antibodies or antigens.

2. Comparison of the sensing device of the present invention with Corning's COR-9018 plate:

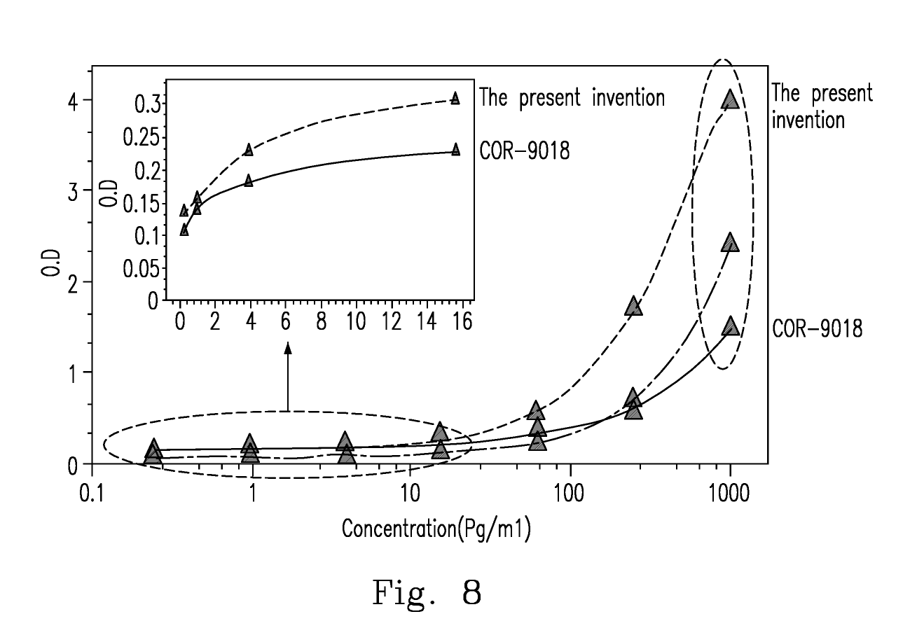

Please refer to FIG. 8, the sensing device of the present invention and Corning's COR-9018 plate are used to carry out ELISA for transforming growth factor-β (TGF-β). TGF-β promotes tumor deterioration, infiltration and metastasis and is a cancer deterioration marker. TGF-β with concentrations far below 1 pg/ml can be detected by ELISA with the sensing device of the present invention. The sensitivity of the sensing device of the present invention is significantly better than Corning's COR-9018 plate.

3. Quantifying the human/mouse transforming growth factor beta 1 using a sandwich ELISA method using a eBioscience human/mouse TGF beta 1 ELISA kit (Cat. No. 88-8350-88) with the sensing device of the present invention and Corning's COR-9018 plate:

Preparing an Assay Plate:

Step 1: Dilute the capture antibody with 1× coating buffer, add 100 μl of diluted capture antibody to each well of each assay plate, seal the assay plates and incubate at 4° C. overnight.

Step 2: After overnight, aspirate the capture antibody liquid in each well. Wash each well with 100 μl of wash buffer five times. Add the liquid along the well wall as much as possible while using an 8-channel pipette to reduce the production of air bubbles. After the last washing step, invert the assay plate on paper towels to remove the residual wash buffer.

Step 3: Add 100 μl of blocking buffer (1X assay diluent) to each well, and place the assay plate at room temperature for at least one hour.

Step 4: Aspirate the liquid in each well. Then, wash each well with 100 μl of wash buffer five times.

ELISA Process:

Step 1: Standards and samples: Serially dilute the standards from the highest concentration of 1000 pg/ml to seven concentrations, where the last is blank. Add 100 μl of the standards and samples (in 1× assay diluent) of each concentration to each well. Each standard and samples are tested in triplicate. Place the assay plate at room temperature for at least two hours after adding the standards and samples.

Step 2: Determination: Aspirate the liquid in each well. Wash each well with 100 μl of wash buffer five times. Add 100 μl of diluted detection antibodies (in 1X assay diluent) of each concentration to each well. Place the assay plate at room temperature for one hour.

Step 3: Binding avidin-horseradish peroxidase (Avidin-HRP): Aspirate the liquid in each well. Wash each well with 100 μl of wash buffer five times. Add 100 μl of diluted Avidin-HRP (in 1× assay diluent) to each well. Place the assay plate away from light at room temperature for 30 minutes.

Step 4: Adding TMB substrate (Substrate solution): Aspirate the liquid in each well. Wash each well with 100 μl of wash buffer seven times. Add 100 μl of substrate to each well. Place the assay plate away from light at room temperature for 15 minutes for coloring. (Add wash buffer to each well and soak for 1-2 minutes before washing.)

Step 5: Add 50 μl Stop Solution to each well. Measure the results using an ELISA analyzer at 450 nm absorption wavelength and calibrate at 630 nm wavelength.

Note: Measure results using an ELISA analyzer within 15-30 minutes after the reaction stops.

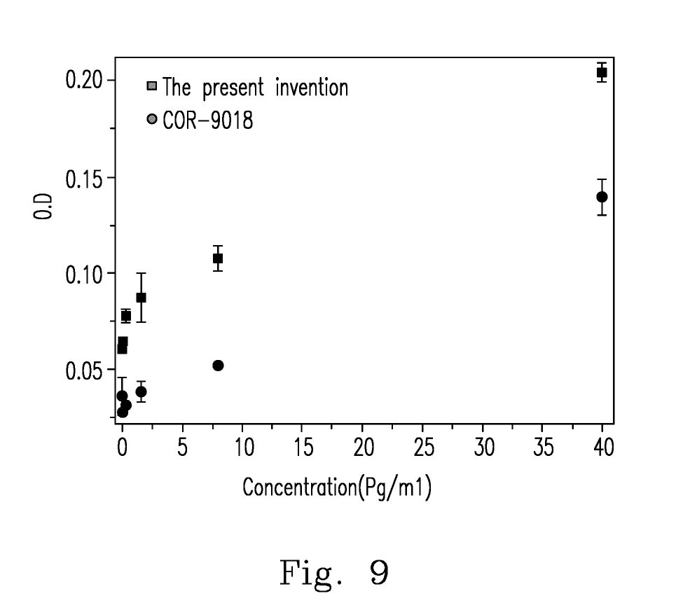

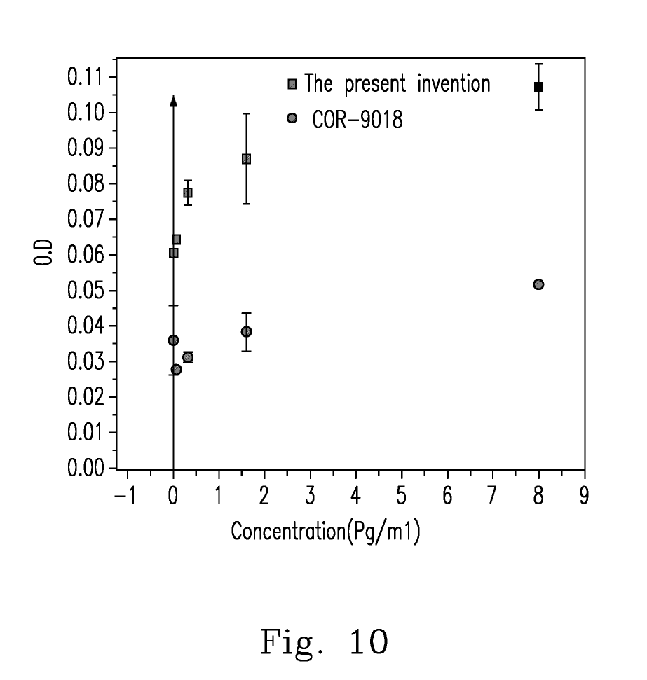

Please refer to FIGS. 9-10, wherein the lowest detection concentration of Corning's COR-9018 plate is 8 pg/mL, while the lowest detection concentration of the sensing device of the present invention (96 well microplate in this embodiment) is 0.064 pg/mL. The entire TGF-β family has nine cysteines that are conserved among its family. Eight form disulfide bonds within the protein to create a cysteine knot structure characteristic of the TGF-β superfamily. The ninth cysteine forms a disulfide bond with the ninth cysteine of another TGF-β protein to produce a dimer. Many other conserved residues in TGF-β are thought to form secondary structures through hydrophobic interactions. Thus, the structural properties of these types of proteins and the structural properties of the surfaces of gold nanoparticles cause these types of proteins to adsorb onto the surfaces of the gold nanoparticles effectively through the ligand bond binding force. Therefore, the noise can be reduced at low concentrations and the overall signal sensitivity can be improved.

4. Quantifying the human/mouse transforming growth factor beta 1 using a sandwich ELISA method using a eBioscience human/mouse TGF beta 1 ELISA kit (Cat. No. 88-8350-88) with the sensing device of the present invention and Corning's COR-9018 plate:

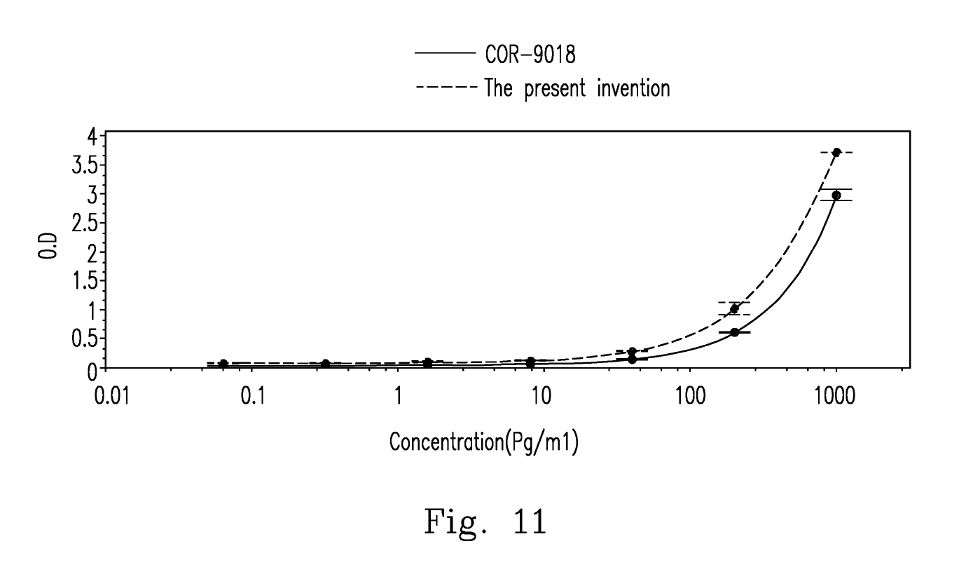

Please refer to FIG. 11 and Table 1, wherein FIG. 11 shows the data in Table 1. In the experiment, the TGF-beta 1 concentration detection limitation of the sensing device of the present invention can achieve 0.064 pg/ml, whereas among commercial assay kit plastic plates, the TGF-beta 1 concentration detection limitation of Corning's COR-9018 plate can only achieve 8 pg/mL. The concentration detection limitation of the sensing device of the present invention exceeds that of the conventional plate by 100 times (0.064 pg/ml: 8 pg/ml).

TABLE 1

| TGF-beta 1 concentration | The sensing device of the | |
|---|---|---|
| (pg/ml) | present invention | COR-9018 |
| 1000 | 3.7064 | 2.9043 |
| 200 | 1.0155 | 0.6148 |
| 40 | 0.27935 | 0.1326 |
| 8 | 0.12365 | 0.0516 |
| 1.6 | 0.09985 | 0.042 |
| 0.32 | 0.0786 | 0.0302 |
| 0.064 | 0.0706 | 0.0275 |
| Blank control group | 0.06655 | 0.0429 |

5. Quantifying the C-Reactive Protein (CRP) using a sandwich ELISA method using a commercial C-Reactive Protein ELISA kit with the sensing device of the present invention and Corning's COR-9018 plate:

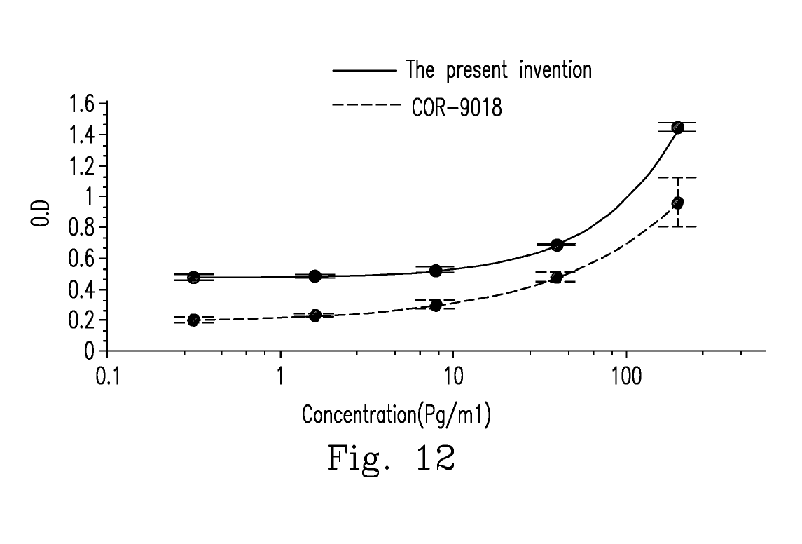

Please refer to FIG. 12 and Table 2, wherein FIG. 12 shows the data in Table 2. The CRP concentration detection limitation of the sensing device of the present invention can achieve 0.32 pg/ml, whereas among commercial assay kit plastic plates, the concentration detection limitation of Corning's COR-9018 plate can only achieve 8 pg/mL. The concentration detection limitation of the sensing device of the present invention exceeds that of the conventional plate by 25 times (0.32 pg/ml: 8 pg/ml).

TABLE 2

| CRP concentration | The sensing device of the | |
|---|---|---|
| (pg/ml) | present invention | COR-9018 |
| 200 | 0.9585 | 1.44165 |
| 40 | 0.4772 | 0.6867 |
| 8 | 0.29615 | 0.5217 |
| 1.6 | 0.2284 | 0.4813 |
| 0.32 | 0.2035 | 0.47285 |
| Blank control group | 0.19125 | 0.48395 |

6. Comparison of carrying out ELISA using the sensing device with four different materials of (1) a glass substrate having gold nanoparticles, wherein surfaces of the gold nanoparticles are modified with amino silane; (2) a glass substrate having gold nanoparticles; (3) a glass substrate modified with amino silane directly; and (4) a glass substrate only:

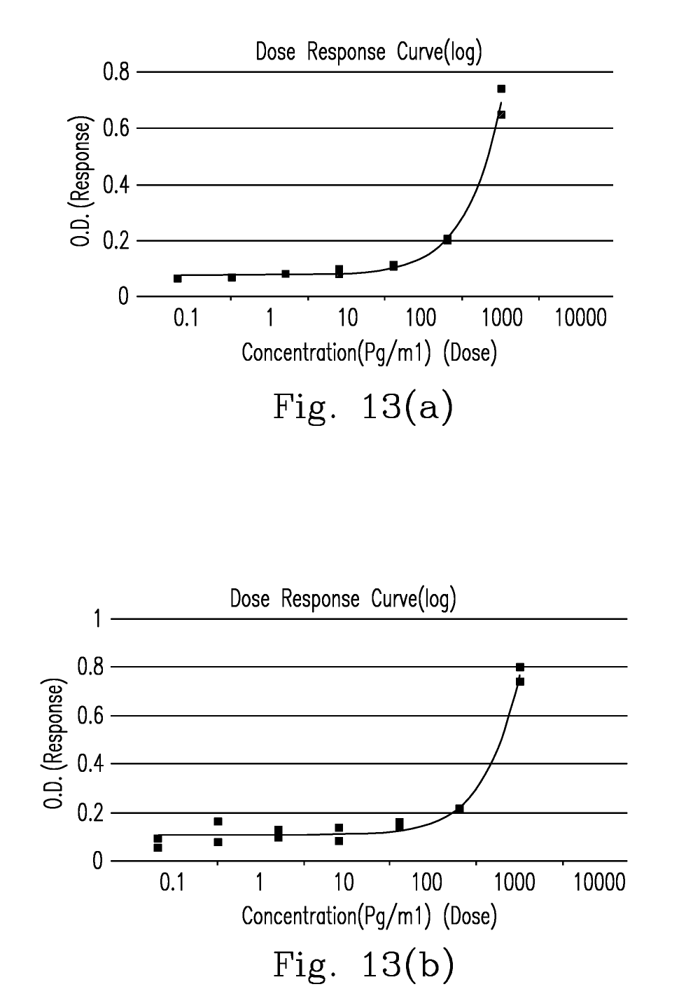

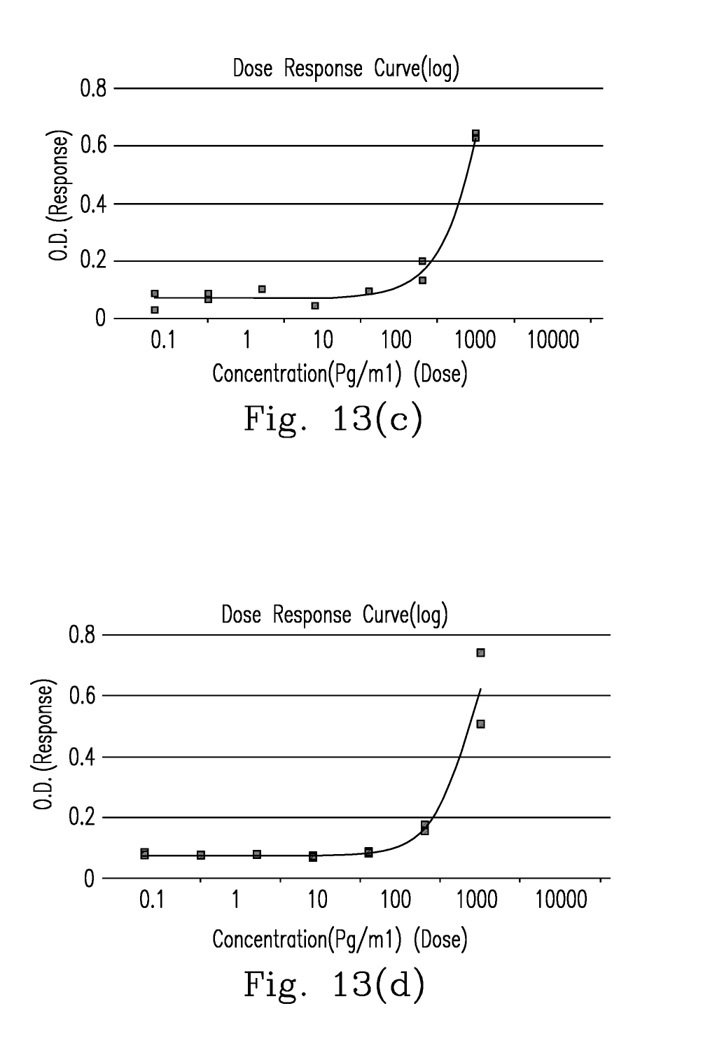

Please refer to FIGS. 13(a)-13(d). Carry out experiments using a eBioscience human/mouse TGF beta 1 ELISA kit (Cat. No. 88-8350-88) with the sensing device where the surfaces of the gold nanoparticles are modified with amino silane, whose signal sensitivity can achieve 0.064 pg/ml, whereas the signal sensitivity of a general plastic plate can only achieve 8 pg/mL. Thus, the signal sensitivity of the sensing device where the surfaces of the gold nanoparticles are modified with amino silane exceeds that of the conventional plate by 100 times (0.064 pg/ml: 8 pg/ml). When a glass substrate of a the sensing device only has gold nanoparticles, and the gold nanoparticles are not modified with amino silane, its signals are relatively unstable, change irregularly, and its confidence declines at low concentrations. Thus, its signal sensitivity is not as good as the sensing device where the surfaces of the gold nanoparticles are modified with amino silane. The experiments showed that the sensing devices with the material of a glass substrate modified with amino silane directly and the glass substrate only did not have an effect to improve sensitivity.

7. Comparison of carrying out ELISA using the sensing device with two different materials of a glass substrate having gold nanoparticles, wherein the surfaces of the gold nanoparticles are modified with amino silane, and a glass substrate only at low concentrations (concentration detection limitation test):

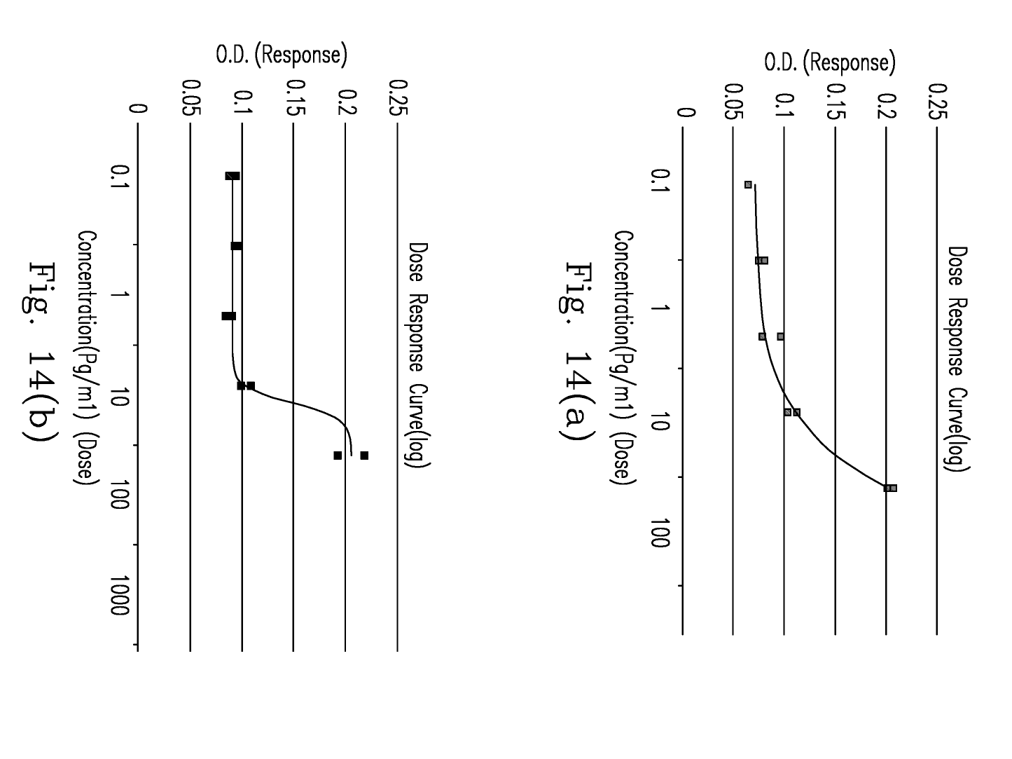

Please refer to FIG. 14(a) and FIG. 14(b), wherein the concentration detection limitation of the sensing device with the materials of a glass substrate having gold nanoparticles is much better than that of the sensing device with the materials of a glass substrate only.

8. A protein chip applied to the sensing chip of the present invention:

Change the reaction zone on the general protein chips to a reaction zone with nanoparticle arrays and an organic molecular layer. The standard of the protein chip is 75 mm*25 mm, and the reaction zone is 60 mm*21 mm.

9. A gene chip applied to the sensing chip of the present invention:

Change the reaction zone on the general gene chips to a reaction zone with nanoparticle arrays and an organic molecular layer. Determine nucleic acid sequences using nucleic acid probe hybridization. Then, generate a graph document after reading the gene chip using a scanner. After the image recognition processes of gridding, spot identifying and noise filtering, fluorescence signal intensity values are obtained and outputted in the form of a list.

### Embodiments

1. A sensing chip for sensing a first organic molecule in a sample, comprising a substrate having a plurality of nanoparticles spaced apart thereon and a plurality of local substrate surfaces located among the plurality of nanoparticles; a silane disposed on a specific one the plurality of local substrate surfaces; and a second organic molecule disposed on a surface of a specific one of the plurality of nanoparticles, wherein there is a specificity between the second and the first organic molecules.
2. The sensing chip of Embodiment 1, wherein the sensing chip is configured to be detachably disposed on a base element having a through hole for closing the through hole at one end thereof to form a sensing device.
3. The sensing chip of any one of Embodiments 1-2, wherein the sensing device is further assembled in a frame to sense the first organic molecule in the sample.
4. The sensing chip of any one of Embodiments 1-3, wherein the second organic molecule is directly disposed on the surface of the specific one of the plurality of nanoparticles.
5. The sensing chip of any one of Embodiments 1-4, wherein the sensing chip is one of a protein chip and a gene chip.
6. The sensing chip of any one of Embodiments 1-5, wherein the first organic molecule is a protein.
7. The sensing chip of any one of Embodiments 1-6, wherein the protein is one of an antigen and an antibody.
8. The sensing chip of any one of Embodiments 1-7, wherein the plurality of nanoparticles includes a metal.
9. A sensing chip for sensing a first organic molecule in a sample, comprising a substrate having a plurality of nanoparticles spaced apart thereon and a plurality of local substrate surfaces located among the plurality of nanoparticles; a second organic molecule disposed on a surface of a specific one of the plurality of nanoparticles, wherein there is a specificity between the second and the first organic molecules; and a modifier disposed on a specific one of the plurality of local substrate surfaces, and dispelling the second organic molecule away from the specific one of the plurality of local substrate surfaces.
10. The sensing chip of Embodiment 9, wherein the modifier is an aminosilane.
11. The sensing chip of any one of Embodiments 9-10, wherein the sensing chip is configured to be detachably disposed on a base element having a through hole for closing the through hole at one end thereof to form a sensing device.
12. The sensing chip of any one of Embodiments 9-11, wherein the sensing device is further assembled in a framework to sense the first organic molecule in the sample.
13. The sensing chip of any one of Embodiments 9-12, wherein the second organic molecule is directly disposed on the surface of the specific one of the plurality of nanoparticles.
14. The sensing chip of any one of Embodiments 9-13, wherein the sensing chip is one of a protein chip and a gene chip.
15. A sensing method for sensing a first organic molecule in a sample, comprising steps of providing a sensing chip including a substrate, a plurality of spaced apart nanoparticles on the substrate and a plurality of local substrate surfaces located among the plurality of nanoparticles; disposing a silane on a specific one of the plurality of local substrate surfaces, and disposing a second organic molecule on a surface of a specific one of the plurality of spaced apart nanoparticles, wherein the silane enhances an adsorptivity of the second organic molecule to the specific one of the plurality of spaced apart nanoparticles; and there is a specificity between the second and the first organic molecules.
16. The sensing method of Embodiment 15, further comprising steps of performing an Enzyme-linked immunosorbent assay after disposing the second organic molecule on the surface of the specific one of the plurality of spaced apart nanoparticles.
17. The sensing method of any one of Embodiments 15-16, wherein the second organic molecule is directly disposed on the surface of the specific one of the plurality of spaced apart nanoparticles; and the second organic molecule is a protein.
18. The sensing method of any one of Embodiments 15-17, wherein the protein is one of an antigen and an antibody.
19. The sensing method of any one of Embodiments 15-18, wherein the sensing chip is one of a protein chip and a gene chip.
20. The sensing method of any one of Embodiments 15-19, wherein the plurality of spaced apart nanoparticles are made of a metal.

What is claimed is:

1. A sensing chip for sensing a first organic molecule in a sample, comprising:
a substrate having a plurality of nanoparticles spaced apart thereon and a plurality of local substrate surfaces located among the plurality of nanoparticles;
a silane disposed on a specific one the plurality of local substrate surfaces; and
a second organic molecule disposed on a surface of a specific one of the plurality of nanoparticles, wherein there is a specificity between the second and the first organic molecules.

2. The sensing chip according to claim 1, wherein the sensing chip is configured to be detachably disposed on a base element having a through hole for closing the through hole at one end thereof to form a sensing device.

3. The sensing chip according to claim 2, wherein the sensing device is further assembled in a frame to sense the first organic molecule in the sample.

4. The sensing chip according to claim 1, wherein the second organic molecule is directly disposed on the surface of the specific one of the plurality of nanoparticles.

5. The sensing chip according to claim 1, wherein the sensing chip is one of a protein chip and a gene chip.

6. The sensing chip according to claim 1, wherein the first organic molecule is a protein.

7. The sensing chip according to claim 6, wherein the protein is one of an antigen and an antibody.

8. The sensing chip according to claim 1, wherein the plurality of nanoparticles includes a metal.

9. A sensing chip for sensing a first organic molecule in a sample, comprising:
a substrate having a plurality of nanoparticles spaced apart thereon and a plurality of local substrate surfaces located among the plurality of nanoparticles;
a second organic molecule disposed on a surface of a specific one of the plurality of nanoparticles, wherein there is a specificity between the second and the first organic molecules; and
a modifier disposed on a specific one of the plurality of local substrate surfaces, and dispelling the second organic molecule away from the specific one of the plurality of local substrate surfaces.

10. The sensing chip according to claim 9, wherein the modifier is an aminosilane.

11. The sensing chip according to claim 9, wherein the sensing chip is configured to be detachably disposed on a base element having a through hole for closing the through hole at one end thereof to form a sensing device.

12. The sensing chip according to claim 11, wherein the sensing device is further assembled in a framework to sense the first organic molecule in the sample.

13. The sensing chip according to claim 9, wherein the second organic molecule is directly disposed on the surface of the specific one of the plurality of nanoparticles.

14. The sensing chip according to claim 9, wherein the sensing chip is one of a protein chip and a gene chip.

15. A sensing method for sensing a first organic molecule in a sample, comprising steps of:
providing a sensing chip including a substrate, a plurality of spaced apart nanoparticles on the substrate and a plurality of local substrate surfaces located among the plurality of nanoparticles;
disposing a silane on a specific one of the plurality of local substrate surfaces, and
disposing a second organic molecule on a surface of a specific one of the plurality of spaced apart nanoparticles, wherein the silane enhances an adsorptivity of the second organic molecule to the specific one of the plurality of spaced apart nanoparticles; and there is a specificity between the second and the first organic molecules.

16. The sensing method according to claim 15, further comprising steps of:
performing an Enzyme-linked immunosorbent assay after disposing the second organic molecule on the surface of the specific one of the plurality of spaced apart nanoparticles.

17. The sensing method according to claim 15, wherein:
the second organic molecule is directly disposed on the surface of the specific one of the plurality of spaced apart nanoparticles; and
the second organic molecule is a protein.

18. The sensing method according to claim 17, wherein the protein is one of an antigen and an antibody.

19. The sensing method according to claim 15, wherein the sensing chip is one of a protein chip and a gene chip.

20. The sensing method according to claim 15, wherein the plurality of spaced apart nanoparticles are made of a metal.
